(19) United States
(12) Patent Application Publication
(10) Pub. No.: US 2019/0061205 A1
(43) Pub. Date: Feb. 28, 2019

(54) PU THIN COVERS WITH WATERPROOF AND MOISTURE PERMEABLE FUNCTION

(71) Applicant: MING-YI LIN, ZHUSHAN TOWNSHIP (TW)

(72) Inventors: MING-YI LIN, ZHUSHAN TOWNSHIP (TW); CHU-YUAN CHEN, ZHUSHAN TOWNSHIP (TW)

(21) Appl. No.: 16/109,636

(22) Filed: Aug. 22, 2018

(30) Foreign Application Priority Data
Aug. 30, 2017 (TW) 106129424
Aug. 30, 2017 (TW) 106212810

(51) Int. Cl. B29C 41/00; B29C 41/14; B29C 41/20; A41D 19/00; A41B 11/00; A41D 31/00; A61F 6/04

(52) U.S. Cl. CPC B29C 41/003; B29C 41/14; B29C 41/20; B29K 2075/00; A41B 11/00; A41D 31/0016; A61F 6/04; A41D 19/0055

(57) ABSTRACT

This invention relates to a PU thin covers with waterproof and moisture permeable function, the hydrophilicity of the PEG structure can diffuse the water molecules from the inside of the PU then covers to the outside of the PU thin covers, and then the water molecules at the outer surface of the PU thin covers will be volatilize to achieve the effect of high moisture permeability. Since the PU thin covers containing PEG structure has no perforations, the external water and air cannot directly penetrate into the inside of the PU thin covers to achieve high waterproof effect. Moreover, the PU materials containing PEG structure can use various special manufacturing methods to make the PU thin covers have the best waterproofness, moisture permeability, structural strength, lightness, low cost, low pollution, high environmental value and various functions can be customized.

### CROSS-REFERENCE TO RELATED APPLICATION

This application claims the priority benefit of Taiwan application No. 106212810 filed on Aug. 30, 2017, and application Ser. No. 10/612,9424 filed on Aug. 30, 2017. The contents of these prior applications are hereby incorporated by reference in its entirety.

### BACKGROUND OF THE INVENTION

### 1. Field of the Invention

This invention relates to a PU thin covers with waterproof and moisture permeable function, particularly to a PU thin covers made of a special PU materials containing PEG structure and a variety of special manufacturing methods to achieve high water resistance (no ventilation pores), high moisture permeability, high structural strength, light weight, low cost, low pollution, high environmental protection, customizable and other effects.

### 2. Description of the Prior Art

At present, the PU cover products sold in the market are mainly divided into three types. The first PU cover product is to take part of the cloth glove (only the inner surface of the fingers and the palm, not including other parts) to coat the PU to form a composite non-slip glove with only part of the outer surface of the glove covered with PU (such as common work gloves). Because the permeability of the foregoing PU is very poor, and the cloth glove is too thick and too compact, the foregoing PU will be very difficult to penetrate into the inner side of the cloth glove, and only a thick PU film will be formed on the outer surface of the cloth glove, so that the foregoing PU film very easy to fall off. During the drying process of the foregoing PU film which is formed on the surface of the cloth glove, the high temperature will cause the solvent gas of the PU to be rushed out from the surface of the PU film to form a plurality of holes, so that the foregoing PU film can increase the friction force, but this also causes the foregoing PU film to have very poor water resistance and appearance, and does not have any moisture permeable effect; for this reason, the glove is very odorous and bacteria-producing when used. Moreover, the thicker the PU film, the worse its elasticity, so the application of the first PU cover product is very limited.

The second PU cover product is pre-formed into a PU flat film, and the PU flat film is cut, then the PU film is adhered with other cloth pieces by high-frequency hot pressing bonding method to form a composite glove. The disadvantages of the second PU cover product are high processing cost, poor structural strength, inconsistent elasticity, very ugly appearance, not environmental protection, inconsistent feeling when worn, sticky feeling and astringent feeling. Because only some parts of the glove have PU characteristics, so that the second PU cover product is very poor in waterproof and moisture permeability.

The third PU cover product is pre-formed into a PU flat film, and the PU flat film is cut, then the two PU films are adhered together by high-frequency hot pressing bonding method to form a PU glove. The disadvantages of the third PU sleeve product are high processing cost, poor structural strength at the joint position, not ergonomic when worn, sticky feeling and astringent feeling.

In addition, the Taiwan Patent No. M543046, “MULTIFUNCTION PU FILM COVER WITH WATERPROOF AND MOISTURE PERMEABLE FUNCTION” which is filed by the inventor, although the cover is integrally formed from the PU material containing PEG structure and has waterproof and moisture permeable functions, there are two problems to be solved. First, when the water is used as the PU dilution solvent, the PU cover is very difficult to be dried, and thus the manufacturing cost is very high and the production efficiency is very low (the drying time is too long and the power consumption is too large). Second, when the prior art uses the oily PU dilution solvent, the PU cover is directly dried, and a large amount of volatile solvent generated during the drying process causes high pollution, so if mass production is required, it have to face a lot of legal issues and environmental issues. Moreover, the foregoing PU cover also has functional expansion limitations, so it cannot be functionally adjusted according to all kinds of special needs, such as adding extra strength, rigidity, elasticity, heat insulation, conductivity, etc.

For this reason, the inventor of this invention, having much experience in designing and manufacturing PU thin covers and its related products, understands and researches the problem of the foregoing PU covers and hence devised this invention.

### SUMMARY OF THE INVENTION

The objective of this invention is to solve various problems existing in the foregoing PU covers, utilizing PU (polyurethane) materials containing PEG (polyethylene glycol) structure and the special method for manufacturing various PU thin covers and PU composite thin sleeve to create a new PU thin covers with the best water resistance (no ventilation pores), moisture permeability, structural strength, light weight, low cost, low pollution, high environmental protection, customizable and other functions.

The PU thin covers with waterproof and moisture permeable function in the present invention includes at least a PU thin cover which is integrally formed from a PU material containing PEG structure, said PU thin cover is provided with an opening, a groove is formed in the opening, the inner surface of the PU thin cover is provided with an rough surface to increase the dry comfort of wearing; said PU material containing PEG structure is diluted with an oily dilution solvent, said PU thin cover may be manufactured by at least one dipping process, at least one cover forming process in water and at least one drying process.

The PU thin covers with waterproof and moisture permeable function of present invention, among which said PU thin cover can be made into a composite PU thin cover according to requirements, which includes at least a inner cover and a PU coating. Said inner cover is provided with a plurality of fine mesh holes, said PU coating is integrally formed on the inner surface and the outer surface of said inner cover by PU material containing a PEG structure, said PU coating may be completely coated on the inner cover or only covered in a part of the inner cover, the inner surface of said PU coating is provided with an rough surface to increase the dry comfort of wearing.

The foregoing composite PU thin cover of present invention, among which said PU material containing PEG structure may be added to the oily dilution solvent to form a PU diluent, and then preparing a mold for mounting said inner cover, said mold is provided with a matte surface, and the composite PU thin cover with waterproof and moisture permeable function is made through at least one dipping process, at least one PU coating forming process in water and at least one drying process.

The foregoing composite PU thin cover of present invention, among which said PU material containing PEG structure may be added to the aqueous dilution solvent to form a PU diluent, and then preparing a mold for mounting said inner cover, said mold is provided with a matte surface, and the composite PU thin cover with waterproof and moisture permeable function is made through at least one dipping process and at least one drying process.

The foregoing composite PU thin cover of present invention, among which a PU film may be pre-manufactured on the mold by using said PU diluent before the inner cover is arranged on the mold, and then to arrange the inner coverer on the PU film to increase the thickness of the PU coating on the inner surface of the inner cover.

### BRIEF DESCRIPTION OF DRAWINGS

This invention will be better understood by referring to the accompanying drawings, wherein:

FIG. 1 is a three-dimensional view of the first embodiment in the present invention;

FIG. 2 is an sectional view of A-A shown in FIG. 1;

FIG. 3 is a front view of the second embodiment in the present invention; and

FIG. 4 is an sectional view of B-B shown in FIG. 3.

### DETAILED DESCRIPTION OF THE PREFERRED EMBODIMENT

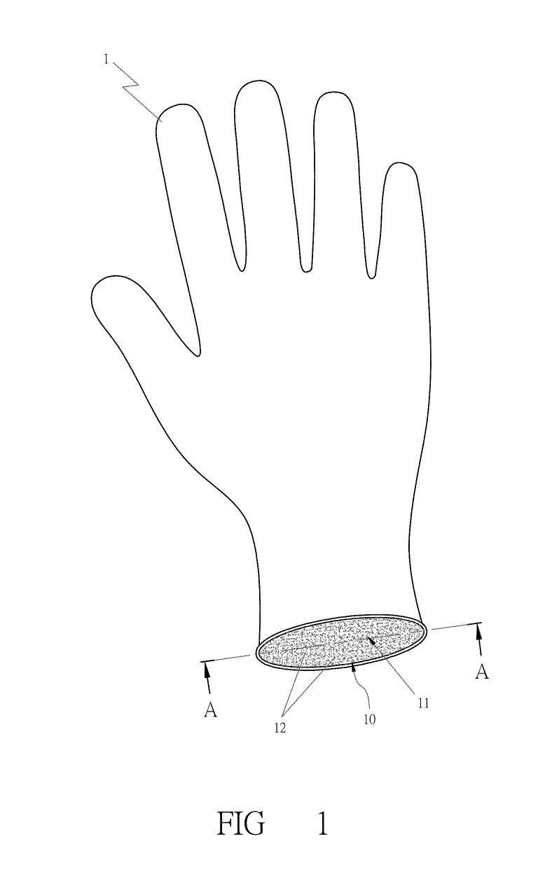

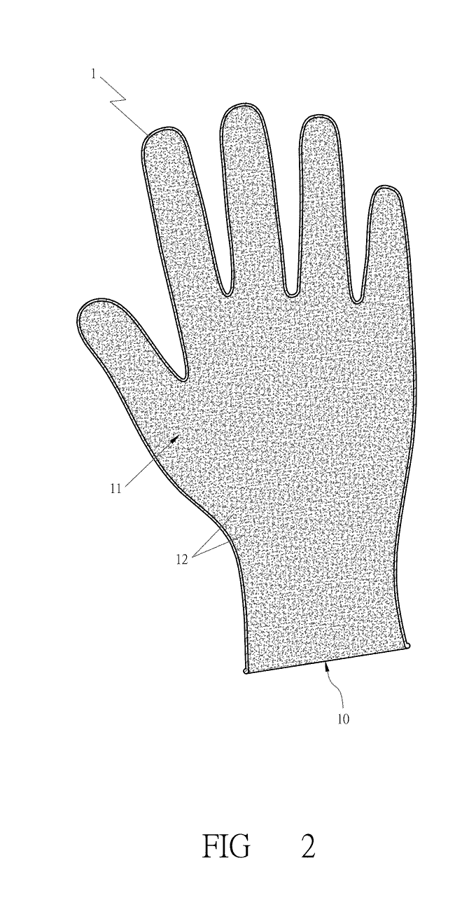

A preferred first embodiment of the PU thin covers with waterproof and moisture permeable function in the present invention, as shown in FIGS. 1 and 2, which includes at least a cover 1 as main components. The cover 1 is integrally formed from a PU (polyurethane) material containing PEG (polyethylene glycol) structure, it has the material properties of PU such as stretchability, tear resistance, elasticity, etc., and the hydrophilicity of the PEG structure can diffuse the water molecules from the inside of the cover 1 to the outside of the cover 1, and then the water molecules at the outer surface of the cover 1 will be volatilize to achieve the effect of high moisture permeability. Moreover, since the PU cover 1 containing PEG structure has no perforations, the external water and air cannot directly penetrate into the inside of the cover to achieve high waterproof effect.

As shown in FIG. 1, said cover 1 is provided with an opening 10, a groove 11 is formed in the opening 10, the shape and the size of the groove 11 may be adjusted according to various requirements for the user to wear. As shown in FIG. 2, the inner surface of the cover 1 is provided with an rough surface (matte) 12 to increase the dry comfort of wearing. Said cover 1 may be a glove, a foot cover, a head cover, a finger sleeve, a joint cover, an arm cover, a leg cover, a condom, a garment, a trousers or other body-worn covers.

Said PU material containing PEG structure is diluted with an oily dilution solvent, such as Dimethylformamide (DMF), a mixed solvent containing Dimethylformamide, and the like. Said cover 1 is manufactured by at least one dipping process, at least one cover forming process in water and at least one drying process.

Said dipping process is to directly dip the mold with matte surface into the PU diluent, and uniformly covers the PU diluent on the outer surface of the mold. The fine gap of the matte surface of the mold can make the PU located in said fine gap break away from the fine gap due to the shrinkage of the PU material during the drying process of the cover 1, so that the contact area of the mold outer surface with the PU can be effectively reduced and the cover 1 can be more easily demolded (the ultra-thin cover with a thickness of about 0.01 mm can be demoulded smoothly). Said mold will form a rough surface 12 on the inner surface of the cover 1, the rough surface 12 can reduce the contact area between the inner surface of the cover 1 and the user's skin, so that the cover 1 can be worn more dry and comfortable, and the user will not feel uncomfortable even if he/she wears it for a long time (if said mold is provided with a smooth outer surface, the PU thin cover formed on the smooth outer surface will adhere to the mold, so that the PU thin cover product will not easy to demould, and the user will feel sticky and astringent when wearing the PU thin cover product). Said mold may be made of metal, wood, ceramic, or other material that can be made into a mold.

Said cover forming process in water is to place said mold with surface covering PU diluent in water to form a cover. Since water can exchange part of the oily dilution solvent of the PU diluent into water, and the PU is incompatible with water, so that the PU can be separate out in water to achieve rapid shaping effect. Moreover, since said cover forming process in water has exchanged a large amount of oily dilution solvent into water, the oily dilution solvent exchanged into the water can be retrieved and reused, and the amount of oily dilution solvent volatilized in the drying process can be reduced to effectively reduce the environmental pollution caused by oily dilution solvent.

Said drying process is to dry the mold with the cover 1 in an oven to complete the manufacture of the cover 1. Since the foregoing process has shaped the cover and reduced a large amount of oily dilution solvent in the cover, the drying time of the cover can be reduced, the difficulty of drying can be reduced (don't worry about the continuous flow of PU diluent on the mold during the drying process to result in uneven distribution of the thickness of the PU cover), and the erosion of the outer surface of the cover 1 by the oily dilution solvent can be prevented (because the higher the temperature, the faster the evaporation rate of the oily dilution solvent, the rapid evaporation of a large amount of oily dilution solvent will cause the surface of the cover 1 to form an eroded perforation defects, destroying the waterproof function of the cover).

Thus, with the improvement of the foregoing technology, the first embodiment of the PU thin covers in the present invention can achieve high production efficiency, high quality stability, low environmental pollution and low cost.

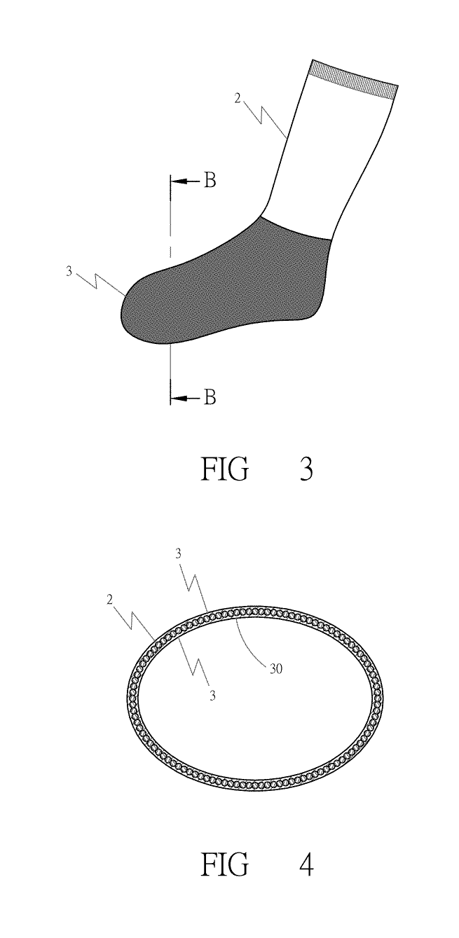

The second embodiment of the PU thin covers with waterproof and moisture permeable function in the present invention, as shown in FIGS. 3 and 4, includes at least an inner cover 2 and a PU coating 3 as main components combined together.

Said inner cover 2 is provided with a plurality of fine mesh holes, the inner cover 2 may be a braided cover woven from artificial chemical fiber, natural fiber, metal fiber, ceramic fiber, glass fiber, etc., or the inner cover 2 may be a plastic cover or a rubber cover with a plurality of mesh holes to provide the PU thin covers additional structure strength, rigidity, elasticity, thermal insulation, electrical conductivity or other functions. Said inner cover 2 may be a glove, a foot cover, a head cover, a finger sleeve, a joint cover, an arm cover, a leg cover, a condom, a garment, a trousers or other body-worn covers.

Said PU coating 3 is integrally formed on the inner surface and the outer surface of said inner cover 2 by PU (polyurethane) material containing a PEG (polyethylene glycol) structure, said PU coating 3 may be completely coated on the inner cover 2 or only covered in a part of the inner cover 2, depending on the needs of use (the embodiment shown in FIG. 3 only coats the PU coating 3 at the lower position of the inner cover 2). The inner surface of said PU coating 3 is provided with an rough surface (matte) 30 to increase the dry comfort of wearing. Said PU coating 3 has the material properties of PU such as stretchability, tear resistance, elasticity, etc., and the hydrophilicity of the PEG structure can diffuse the water molecules from the inside of the PU coating 3 to the outside of the PU coating 3, and then the water molecules at the outer surface of the PU coating 3 will be volatilize to achieve the effect of high moisture permeability. Moreover, since the PU coating 3 containing PEG structure has no perforations, the external water and air cannot directly penetrate into the inside of the PU coating 3 to achieve high waterproof effect.

When manufacturing the second embodiment of the PU thin covers in the present invention, the first step is to add a dilution solvent to said PU material containing PEG structure to form a PU diluent. Said dilution solvent may be an oily dilution solvent (such as DMF (Dimethylformamide), a mixed solvent containing DMF, and the like) or an aqueous dilution solvent (such as pure water or aqueous mixture). Then, preparing a mold for mounting said inner cover 2, said mold is provided with a matte surface. The fine gap of the matte surface of the mold can make the PU located in said fine gap break away from the fine gap due to the shrinkage of the PU material during the drying process of the PU coating 3, so that the contact area of the mold outer surface with the PU can be effectively reduced and the PU coating 3 can be more easily demolded (the ultra-thin cover with a thickness of about 0.01 mm can be demoulded smoothly). Said mold will form a rough surface 30 on the inner surface of the PU coating 3, the rough surface 30 can reduce the contact area between the inner surface of the PU coating 3 and the user's skin, so that the PU thin covers can be worn more dry and comfortable, and the user will not feel uncomfortable even if he/she wears it for a long time (if said mold is provided with a smooth outer surface, the PU thin cover formed on the smooth outer surface will adhere to the mold, so that the PU thin cover product will not easy to demould, and the user will feel sticky and astringent when wearing the PU thin cover product). Said mold may be made of metal, wood, ceramic, or other material that can be made into a mold.

The second step is to arrange said inner cover 2 on the mold and dip the mold with the inner cover 2 into the PU diluent. Since the mesh holes of the inner cover 2 allows the PU diluent to penetrate into the inner surface thereof, the inner surface and the outer surface of the inner cover 2 can uniformly coat the PU diluent. In order to increase the thickness of the PU coating 3 on the inner surface of the inner cover 2, a PU film may be pre-manufactured on the mold by using said PU diluent (the thickness of the PU film may be adjusted by at least one PU film process such as at least one dipping process and at least one drying process) before the inner cover 2 is arranged on the mold, and then to arrange the inner coverer 2 on the PU film and dip the mold with the inner cover 2 into the PU diluent, so that the thickness of the PU coating 3 on the inner surface of the inner cover 2 can be adjusted to the desired thickness.

When the PU diluent is diluted with an oily dilution solvent, the third step is to place said mold in water to form the PU coating 3. Since water can exchange part of the oily dilution solvent of the PU diluent into water, and the PU is incompatible with water, so that the PU can be separate out in water to achieve rapid shaping effect. It should be noted that since the inner cover 2 with mesh holes is not a flat surface, the overall distribution of the PU coating 3 is not uniform (the PU coating 3 located at the mesh holes of the inner cover 2 has the most thickest thickness), so that it will result in inconsistent volatilization rate of the oily dilution solvent of the PU coating 3. If said mold is directly fed into the oven for drying, the PU coating 3 will generate solvent vapor erosion and perforations at the mesh hole position of the inner cover 2, thereby causing the PU coating 3 to form defects and reduce the waterproof effect thereof. Therefore, when the PU diluent is diluted with an oily dilution solvent, it have to use this process, the advantage of the PU coating 3 forming in water is that the PU coating 3 can be quickly shaped and a large amount of oily dilution solvent can be exchanged Into water; moreover, the oily dilution solvent exchanged into the water can be retrieved and reused, and the amount of oily dilution solvent volatilized in the drying process can be reduced to effectively reduce the environmental pollution caused by oily dilution solvent.

It is important to note that when the PU diluent is diluted with an aqueous dilution solvent, the PU coating 3 cannot be formed in water because the water will dissolve and disperse the PU diluent. Moreover, the aqueous dilution solvent will not cause the problem of solvent vapor erosion and perforation during the drying process, so the PU coating 3 can be formed by directly drying in an oven.

The fourth step is to feed the mold into the oven and dry the excess moisture and the residual solvent to complete the PU thin covers. If it is necessary to increase the thickness of the PU coating 3 on the outer surface of the inner cover 2 to get better tensile strength (tear strength) and waterproof function, the manufacturer may repeat the foregoing second step to the fourth step to achieve the purpose.

Said PU materials may be added with various additives to increase the functionality, production efficiency, comfort and commercial value of the PU thin covers, depending on the demand. For example, the addition of silicon oil to the PU material can make the PU thin covers have a slippery feel, assist the PU demould and anti-sticking effect; the addition of silica powder to the PU material can make the PU thin covers have the functions of dry hand, anti-sticking and surface extinction; the addition of hardening agent containing isocyanate group (—N═C═O) to PU material can further strengthen the physical properties and mechanical strength of the PU thin covers; the addition of titanium dioxide to the PU material can make the PU thin covers have the functions of antistatic (electrical conductivity) and dyed white, and the PU thin covers can directly perform the touch operation of the electronic product; the addition of the antibacterial agent to the PU material can inhibit the growth of bacteria and mold; the addition of coloring agent to the PU material can make the PU thin covers have various color changes.

It is worth mentioning that the PU thin covers in the present invention can be adjusted in thickness through a plurality of identical processes according to the use requirements, and the PU thin covers can be made to have a minimum thickness of about 0.01 mm. The moisture permeability of the PU thin covers will not be greatly reduced by the increase of the thickness, and the moisture permeability can reach the level of JIS L1099 B1 test method 15000 (g/m2/24 hr) or more, so that the effect of the thickness on the moisture permeability can be reduced, and the sweat from the skin can be quickly discharged out of the PU thin covers to keep the skin dry. The water resistance of the PU thin covers can reach the level of JIS L1092B test method 10000 (mm-H2O) or more, and various functional inner cover can be arranged according to requirements, so that the PU thin covers can be applied to various industries (such as industrial, commercial, agricultural, aquaculture, medical, beauty, catering or special work) or special needs (such as toughness, steel, elasticity, heat insulation, electrical conductivity, etc.). The PU thin covers also has the anti-solvent property, which allows the user to achieve the effect of blocking the external harmful solvent erosion when worn, and avoids the skin directly being exposed to the harmful solvent. Evidently this invention has tangible benefits and tallies with progressiveness and novelty demanded by patent laws.

While the preferred embodiments of this invention have been described above, it will be recognized and understood that various modifications may be made therein and appended claims are intended to cover all such modifications that may fall within the spirit and scope of the invention.

What is claimed is:

1. A PU thin covers with waterproof and moisture permeable function at least comprising:
a inner cover is provided with a plurality of fine mesh holes;
a PU coating is integrally formed on the inner surface and the outer surface of said inner cover by PU material containing a PEG structure, said PU coating may be completely coated on the inner cover or only covered in a part of the inner cover, the inner surface of said PU coating is provided with an rough surface to increase the dry comfort of wearing;
said PU material containing PEG structure is added to the oily dilution solvent to form a PU diluent, and then preparing a mold for mounting said inner cover, said mold is provided with a matte surface, and the PU thin covers with waterproof and moisture permeable function is made through at least one dipping process, at least one PU coating forming process in water and at least one drying process;
said dipping process is to immerse the mold with the inner cover into the PU diluent, the mesh holes of the inner cover allows the PU diluent to penetrate into the inner surface thereof, so that the inner surface and the outer surface of the inner cover can uniformly coat the PU diluent;
said PU coating forming process in water is to place said mold with surface covering PU diluent in water to form the PU coating and to exchange part of the oily dilution solvent of the PU diluent into water, so that the amount of oily dilution solvent volatilized in the drying process can be reduced to effectively reduce the environmental pollution caused by oily dilution solvent.

2. The PU thin covers with waterproof and moisture permeable function according to claim 1, wherein said inner cover is a braided cover.

3. The PU thin covers with waterproof and moisture permeable function according to claim 1, wherein said inner cover may be a plastic cover or a rubber cover with a plurality of mesh holes.

4. The PU thin covers with waterproof and moisture permeable function according to claim 1, wherein a PU film is pre-manufactured on the mold by using said PU diluent before the inner cover is arranged on the mold, and then to arrange the inner coverer on the PU film to increase the thickness of the PU coating on the inner surface of the inner cover.

5. The PU thin covers with waterproof and moisture permeable function according to claim 4, wherein said inner cover is a braided cover.

6. The PU thin covers with waterproof and moisture permeable function according to claim 4, wherein said inner cover may be a plastic cover or a rubber cover with a plurality of mesh holes.

7. A PU thin covers with waterproof and moisture permeable function at least comprising:
a inner cover is provided with a plurality of fine mesh holes;
a PU coating is integrally formed on the inner surface and the outer surface of said inner cover by PU material containing a PEG structure, said PU coating may be completely coated on the inner cover or only covered in a part of the inner cover, the inner surface of said PU coating is provided with an rough surface 30 to increase the dry comfort of wearing;
said PU material containing PEG structure is added to the aqueous dilution solvent to form a PU diluent, and then preparing a mold for mounting said inner cover 2, said mold is provided with a matte surface, and the PU thin covers with waterproof and moisture permeable function is made through at least one dipping process and at least one drying process;
said dipping process is to immerse the mold with the inner cover into the PU diluent, the mesh holes of the inner cover allows the PU diluent to penetrate into the inner surface thereof, so that the inner surface and the outer surface of the inner cover can uniformly coat the PU diluent; and then drying the mold to make the PU thin covers with waterproof and moisture permeable function.

8. The PU thin covers with waterproof and moisture permeable function according to claim 7, wherein said inner cover is a braided cover.

9. The PU thin covers with waterproof and moisture permeable function according to claim 7, wherein said inner cover may be a plastic cover or a rubber cover with a plurality of mesh holes.

10. The PU thin covers with waterproof and moisture permeable function according to claim 7, wherein a PU film is pre-manufactured on the mold by using said PU diluent before the inner cover is arranged on the mold, and then to arrange the inner coverer on the PU film to increase the thickness of the PU coating on the inner surface of the inner cover.

11. The PU thin covers with waterproof and moisture permeable function according to claim 10, wherein said inner cover is a braided cover.

12. The PU thin covers with waterproof and moisture permeable function according to claim 10, wherein said inner cover may be a plastic cover or a rubber cover with a plurality of mesh holes.
